(19) United States
(12) Patent Application Publication
(10) Pub. No.: US 2021/0100434 A1
(43) Pub. Date: Apr. 8, 2021

(54) DISTAL END HOOD AND ENDOSCOPE SYSTEM

(71) Applicant: OLYMPUS CORPORATION, Tokyo (JP)

(72) Inventors: Masahiro Maeda, Tokyo (JP); Kazuki Honda, Tokyo (JP)

(73) Assignee: OLYMPUS CORPORATION, Tokyo (JP)

(21) Appl. No.: 17/123,739

(22) Filed: Dec. 16, 2020

Related U.S. Application Data
(63) Continuation of application No. PCT/JP2018/027055, filed on Jul. 19, 2018.

(51) Int. Cl. A61B 1/00; A61B 1/12

(52) U.S. Cl. CPC A61B 1/00089; A61B 1/126; A61B 1/00186

(57) ABSTRACT

An endoscope system includes an endoscope and a distal end hood. The endo scope includes an insertion portion, a first optical system, a second optical system, an optical component, and a protruding member. The distal end hood includes a frame and a filter including a light transmission body and a support member provided at a non-contact position with the protruding member to support the light transmission body.

### CROSS REFERENCE TO RELATED APPLICATION

This application is a continuation application of PCT/JP2018/027055 filed on Jul. 19, 2018, the entire contents of which are incorporated herein by this reference.

### BACKGROUND OF THE INVENTION

### 1. Field of the Invention

The present invention relates to a distal end hood and an endoscope system.

### 2. Description of the Related Art

Conventionally, an endoscope system has been known in which an endoscope is inserted into a subject to observe the inside of the subject.

A distal end hood may be attached to a distal end portion of the endoscope to ensure a good field of view or to assist in treatment.

For example, a distal end hood used for photodynamic therapy (PDT) is disclosed in Japanese Patent Application Laid-Open Publication No. 2007-20759. In the photodynamic therapy, a photosensitizer is administered to a patient, and the photosensitizer is excited by irradiation of a laser beam to generate active oxygen, thereby treating a lesion. The distal end hood cuts off reflected light of the laser beam, with which the lesion is irradiated, using a laser beam cut-off filter, so that the lesion can be observed.

As another conventional example, a stereoscopic endoscope has been also known which is configured to pick up a right-eye optical image and a left-eye optical image having parallax and to display a stereoscopic image.

### SUMMARY OF THE INVENTION

An endoscope system according to an aspect of the present invention includes an endoscope; and a distal end hood, the endoscope including an insertion portion configured to be inserted into a subject in a longitudinal direction, a first optical system provided at a distal end portion of the insertion portion and configured to observe the subject, a second optical system provided at the distal end portion to be adjacent to the first optical system and configured to observe the subject with parallax with respect to the first optical system, an optical component provided on a distal end side of the first optical system and the second optical system and having light transmission properties, and a protruding member that further protrudes in a distal end direction from a distal end surface of the distal end portion than the optical component, the distal end hood including a frame attached to a periphery of the distal end portion, and a filter provided on an inner peripheral side of the frame, the filter including a light transmission body provided so as to face a distal end surface of the optical component and having light transmission properties, and a support member provided at a non-contact position with the protruding member to support the light transmission body.

A distal end hood according to another aspect of the present invention includes: a frame attached to a periphery of a distal end portion of an endoscope; and a filter provided on an inner peripheral side of the frame. The filter includes: a light transmission body having light transmission properties and provided so as to face a distal end surface of an optical component of the endoscope provided on a distal end side of a first optical system and a second optical system having parallax with each other; and a support member provided at a non-contact position with a protruding member of the endoscope to support the light transmission body, the protruding member further protruding in a distal end direction from the distal end portion than the optical component.

### BRIEF DESCRIPTION OF THE DRAWINGS

FIG. 1 is a diagram showing an example of a configuration of an endoscope system according to an embodiment of the present invention;

FIG. 2 is a perspective view showing an example of a distal end portion and a distal end hood of an endoscope in the endoscope system according to the embodiment of the present invention;

FIG. 3 is a schematic cross-sectional view showing an example of the distal end portion and the distal end hood of the endoscope in the endoscope system according to the embodiment of the present invention;

FIG. 4 is a schematic cross-sectional view showing an example of a state where the distal end hood is attached to the distal end portion of the endoscope in the endoscope system according to the embodiment of the present invention;

FIG. 5 is an explanatory diagram illustrating an example of a right-eye optical image and a left-eye optical image in the endoscope system according to the embodiment of the present invention;

FIG. 6 is a perspective view showing an example of a distal end portion and a distal end hood of an endoscope in an endoscope system according to Modification 1 of the embodiment of the present invention;

FIG. 7 is a schematic cross-sectional view showing an example of the distal end portion and the distal end hood of the endoscope in the endoscope system according to Modification 1 of the embodiment of the present invention;

FIG. 8 is a schematic cross-sectional view showing an example of a state where the distal end hood is being attached to the distal end portion of the endoscope in the endoscope system according to Modification 1 of the embodiment of the present invention;

FIG. 9 is a schematic cross-sectional view showing an example of a state where the distal end hood is attached to the distal end portion of the endoscope in the endoscope system according to Modification 1 of the embodiment of the present invention;

FIG. 10 is a schematic cross-sectional view showing an example of a distal end portion, a distal end hood, and a drain port of an endoscope in an endoscope system according to Modification 2 of the embodiment of the present invention; and

FIG. 11 is a front view showing an example of a distal end portion and a distal end hood of an endoscope in an endoscope system according to Modification 3 of the embodiment of the present invention.

### DETAILED DESCRIPTION OF THE PREFERRED EMBODIMENTS

An embodiment of the present invention will be described below with reference to the drawings.

(Configuration)

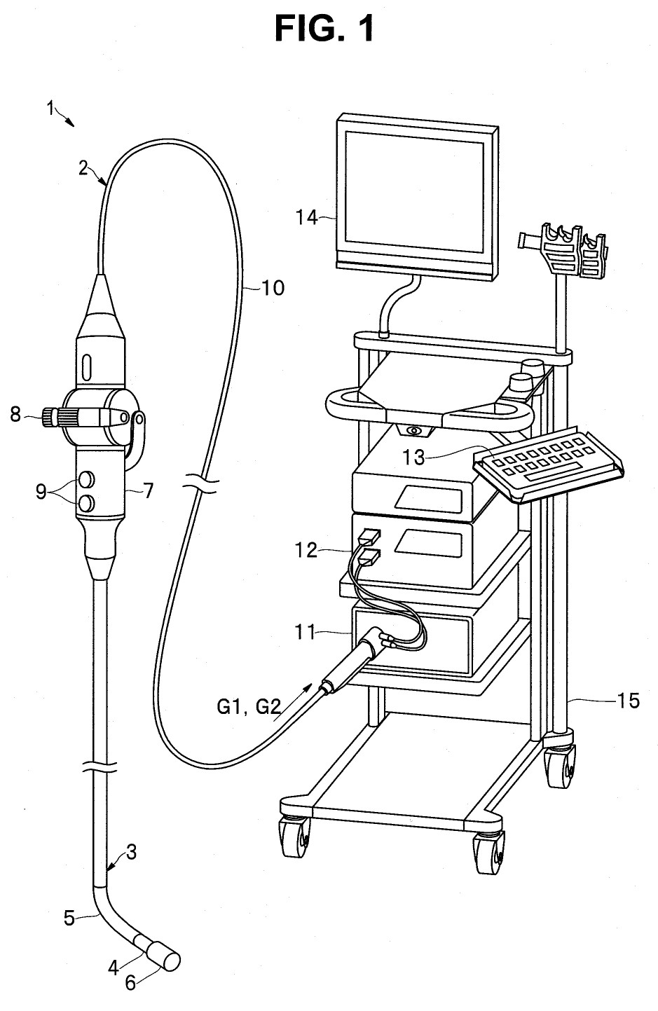

FIG. 1 is a diagram showing an example of a configuration of an endoscope system 1.

As shown in FIG. 1, the endoscope system 1 includes an endoscope 2 capable of stereoscopically observing the inside of a subject in three dimensions.

The endoscope 2 includes an insertion portion 3. The insertion portion 3 has an elongated shape, and is inserted into the subject in a longitudinal direction. The insertion portion 3 includes a distal end portion 4 and a bending portion 5 continuously provided on a proximal end side of the distal end portion 4. A distal end hood 6 is attached to the distal end portion 4.

An operation portion 7 is continuously provided on a proximal end side of the insertion portion 3. The operation portion 7 is provided with a bending operation lever 8 and an operation button 9 that bend the bending portion 5.

A universal cable 10 extends from the operation portion 7 and is connected to a light source apparatus 11 and a video processor 12.

The light source apparatus 11 generates illumination light under control of the video processor 12, and outputs the illumination light to the endoscope 2 via the universal cable 10.

The video processor 12 is provided with an instruction input apparatus 13 such as a keyboard. The video processor 12 is also provided with a water feeding tank and a pump for water feeding or air feeding. The video processor 12 alternately arranges a right-eye optical image G1 and a left-eye optical image G2, which are inputted from the endoscope 2, for each line to generate a stereoscopic image, and outputs the stereoscopic image to a monitor 14.

The monitor 14 displays the stereoscopic image inputted from the video processor 12. A right-eye polarizing filter and a left-eye polarizing filter, which have different polarization directions from each other for each line, are provided on the monitor 14. A user wears polarized eyeglasses and makes a stereoscopic image having left and right parallax.

The video processor 12 and the monitor 14 display a stereoscopic image using a polarization method as an example, but the display method for the stereoscopic image is not limited to the polarization method. The video processor 12 and the monitor 14 may display a stereoscopic image using a liquid crystal shutter method, an autostereoscopic display method, an autostereoscopic viewing method, or another stereoscopic display method, for example.

The light source apparatus 11, the video processor 12, the instruction input apparatus 13, and the monitor 14 are housed in a gantry 15.

Subsequently, the distal end portion 4 and the distal end hood 6 of the endoscope 2 will be described.

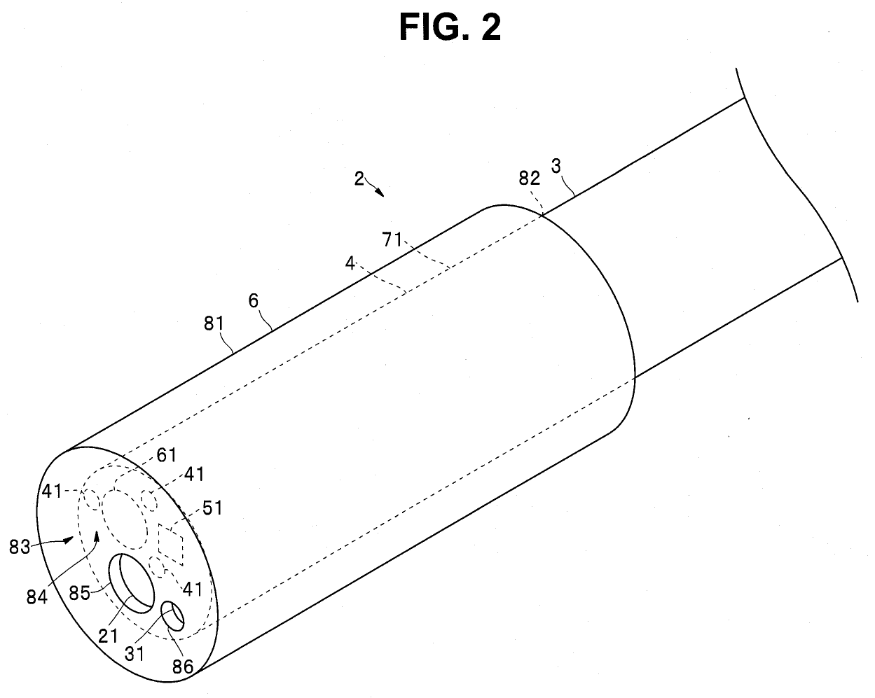

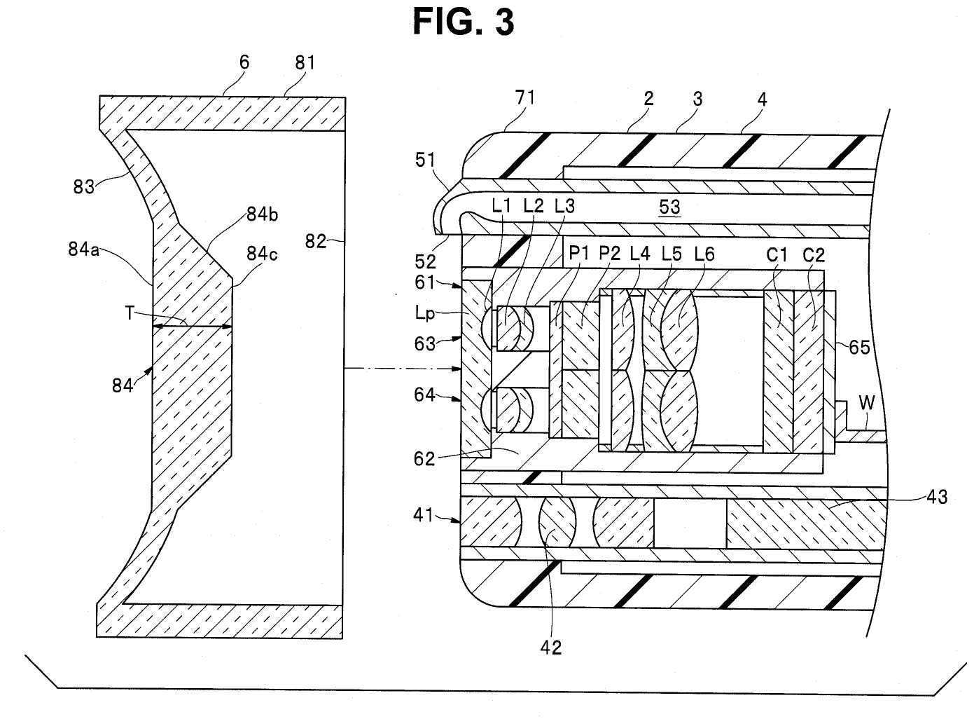

FIGS. 2 and 3 are views showing an example of the distal end portion 4 and the distal end hood 6 of the endoscope 2 in the endoscope system 1. FIG. 2 is a perspective view, and FIG. 3 is a schematic cross-sectional view.

As shown in FIG. 2, the distal end portion 4 includes a treatment instrument insertion opening 21, a fluid feeding opening 31, an illumination unit 41, a nozzle 51 as a protruding member, an image pickup unit 61, and an outer tube 71.

The treatment instrument insertion opening 21 communicates with a treatment instrument insertion port of the operation portion 7 via a treatment instrument conduit provided in the insertion portion 3. The treatment instrument inserted from the treatment instrument insertion port is sent into the subject from the treatment instrument insertion opening 21. The treatment instrument is forceps, for example.

The fluid feeding opening 31 is connected to the video processor 12 via an air/water feeding conduit provided inside the insertion portion 3 and the universal cable 10. When the user operates the operation button 9, the fluid feeding opening 31 delivers a fluid fed from the video processor 12 to a front side.

The illumination unit 41 is arranged to be adjacent to the image pickup unit 61. A plurality of illumination units 41 may be provided around the image pickup unit 61. For example, in the example of FIG. 2, three illumination units 41 are provided around the image pickup unit 61. The illumination unit 41 includes an illumination optical system 42 for irradiating the subject with illumination light. The illumination unit 41 is connected to the light source apparatus 11 by a light guide 43 passed through the universal cable 10. The illumination unit 41 guides the illumination light by the light guide 43, and irradiates the subject with the illumination light via the illumination optical system 42.

As shown in FIG. 3, the nozzle 51 protrudes from a distal end surface of the distal end portion 4, and is provided such that a nozzle opening 52 faces a distal end surface of the image pickup unit 61. The nozzle 51 communicates with an air/water feeding conduit via a nozzle conduit 53. When the user operates the operation button 9, the nozzle opening 52 jets the fluid fed to the nozzle conduit 53 from the video processor 12 onto the distal end surface of the distal end portion 4 to remove dirt from the distal end surface of the image pickup unit 61.

The image pickup unit 61 converts return light of the subject into an image pickup signal. The image pickup unit 61 includes a lens barrel 62, a right-eye optical system 63 as a first optical system, a left-eye optical system 64 as a second optical system, and an image pickup device 65.

The lens barrel 62 is made of a material such as a metal. The lens barrel 62 extends in a length direction of the distal end portion 4 and has a tubular shape.

The right-eye optical system 63 and the left-eye optical system 64 are provided on an inner peripheral side of the lens barrel 62. The right-eye optical system 63 projects the right-eye optical image G1 onto the image pickup device 65. The left-eye optical system 64 projects the left-eye optical image G2 onto the image pickup device 65.

Each of the right-eye optical system 63 and the left-eye optical system 64 includes, in order from the distal end toward the proximal end, a negative lens L1, a positive lens L2, a negative lens L3, a parallel flat plate P1, an optical path change element P2, a positive lens L4, a negative lens L5, a positive lens L6, and a cover glass C1.

The respective negative lenses L1 are provided on an optical component Lp such that optical axes of the negative lenses are separated from each other by a predetermined distance. As the predetermined distance becomes shorter, a vergence angle becomes smaller, whereas as the predetermined distance becomes longer, the vergence angle becomes larger. The predetermined distance is determined in advance according to the type of the subject and the purpose of observation so that the vergence angle is preferable.

The optical component Lp is provided on a distal end side of the right-eye optical system 63 and the left-eye optical system 64, has light transmission properties, and has a plate shape. In FIG. 3, for example, the optical component Lp extends over the right-eye optical system 63 and the left-eye optical system 64. A distal end surface of the optical component Lp has a smooth shape. A proximal end surface of the optical component Lp has a concave shape at a position corresponding to each of the negative lenses L1.

The parallel flat plate P1 and the optical path change element P2 are bonded to each other. In FIG. 3, the parallel flat plate P1 is configured by one member extending over the right-eye optical system 63 and the left-eye optical system 64, but may be provided separately for the right-eye optical system 63 and the left-eye optical system 64. The optical path change element P2 is configured by a reflecting prism, for example. The optical path change element P2 changes an optical path such that each of the right-eye optical image G1 and the left-eye optical image G2 is projected on the image pickup device 65.

The cover glass C1 covers proximal end sides of the right-eye optical system 63 and the left-eye optical system 64.

The image pickup device 65 is provided on a proximal end side of the cover glass C1. The image pickup device 65 includes a cover glass C2. The cover glass C2 is bonded to the cover glass C1. The image pickup device 65 is connected to the video processor 12 by a wiring W. The wiring W passes through the universal cable 10.

The outer tube 71 is made of a material such as resin, and is provided on an outer periphery of the distal end portion 4.

In other words, the image pickup unit 61 is provided at the distal end portion 4 of the insertion portion 3, and includes the first optical system configured to observe the subject, the second optical system provided at the distal end portion 4 to be adjacent to the first optical system and configured to observe the subject with parallax with respect to the first optical system, and the optical component Lp provided on the distal end side of the first optical system and the second optical system and having light transmission properties. The protruding member 51 further protrudes in a distal end direction from the distal end surface of the distal end portion 4 than the optical component Lp.

At least a part of the distal end hood 6 is made of resin having light transmission properties. The distal end hood 6 includes a frame 81, an insertion port 82, a support member 83, a light transmission body 84, a treatment instrument insertion hole 85, and a fluid feeding hole 86 (see FIG. 2).

The frame 81 has a tubular shape, and is attached to the periphery of the distal end portion 4 in such a manner of being externally fitted onto the outer peripheral surface of the distal end portion 4.

The insertion port 82 is provided at the proximal end of the distal end hood 6. The distal end portion 4 is inserted into the insertion port 82.

The support member 83 is provided closer to the distal end side than the insertion port 82, and extends to approach the insertion port 82 toward the inside in a radial direction from an inner peripheral side of the frame 81. The support member 83 has an annular shape. The support member 83 is provided at a non-contact position with the distal end of the nozzle 51 when the light transmission body 84 is provided to face the distal end surface of the optical component Lp, and supports the light transmission body 84. The support member 83 adjusts an angle between the frame 81 and the light transmission body 84 such that the light transmission body 84 comes into parallel contact with the distal end surface of the optical component Lp. For example, when the distal end portion 4 pushes the light transmission body 84, the support member 83 adjusts the angle between the frame 81 and the light transmission body 84 to be orthogonal to the inner peripheral surface in a longitudinal direction of the frame 81.

In addition, the support member 83 has elasticity to allow positioning between the insertion portion 3 and the distal end hood 6 to be performed in a state where the light transmission body 84 is in parallel contact with the distal end surface of the optical component Lp.

In FIG. 3, the support member 83 has a bent plate shape in which the proximal end side surface is convex, but is not limited thereto. For example, the support member 83 may have a tapered tubular shape or a bent plate shape in which the distal end side surface is convex.

The light transmission body 84 is provided on the inner peripheral side of the support member 83. The light transmission body 84 is attached to the distal end surface of the optical component Lp to face each other. All or part of the light transmission body 84 has light transmission properties. The light transmission body 84 includes an incident surface 84a, a main body 84b, and an emission surface 84c.

The incident surface 84a extends inward in the radial direction from an inner edge of the distal end side surface of the support member 83. The return light is incident on the incident surface 84a from the subject.

The main body 84b protrudes in the proximal end direction from the inside of the support member 83. In the example of FIG. 3, the main body 84b has a frustum shape, but is not limited to such a shape. For example, the main body 84b may have a short pillar shape or another shape.

The emission surface 84c is provided at a proximal end of the main body 84b to be parallel to the incident surface 84a. The emission surface 84c has a shape that is in close contact with the entire distal end surface of the optical component Lp. The emission surface 84c has a smooth shape, for example.

A predetermined thickness T is provided between the incident surface 84a and the emission surface 84c. The predetermined thickness T is larger than a thickness of the support member 83, and is more preferably adjusted and determined in advance such that right-eye optical image G1 and the left-eye optical image G2 can be picked up.

Further, a size and a radial position of each of the incident surface 84a and the emission surface 84c are adjusted and determined in advance according to a predetermined angle of view.

The support member 83 and the light transmission body 84 configure a filter.

At the distal end of the distal end hood 6, the treatment instrument insertion hole 85 is provided at a position corresponding to the treatment instrument insertion opening 21.

At the distal end of the distal end hood 6, the fluid feeding hole 86 is provided at a position corresponding to the fluid feeding opening 31.

In other words, the distal end hood 6 includes the frame 81 and the filter. The frame 81 is attached to the periphery of the distal end portion 4 in such a method of being externally fitted onto the outer peripheral surface of the distal end portion 4 of the endoscope 2. The filter includes: the light transmission body 84 provided on the inner peripheral side of the frame 81 so as to face the distal end surface of the optical component Lp of the endoscope 2 provided on the distal end side of the first optical system and the second optical system having parallax with each other, and having light transmission properties; and the support member 83 provided at the non-contact position with the protruding member of the endoscope 2 further protruding in the distal end direction from the distal end portion 4 than the optical component Lp to support the light transmission body 84.

(Operation)

An operation of the endoscope system 1 of the embodiment will be described below.

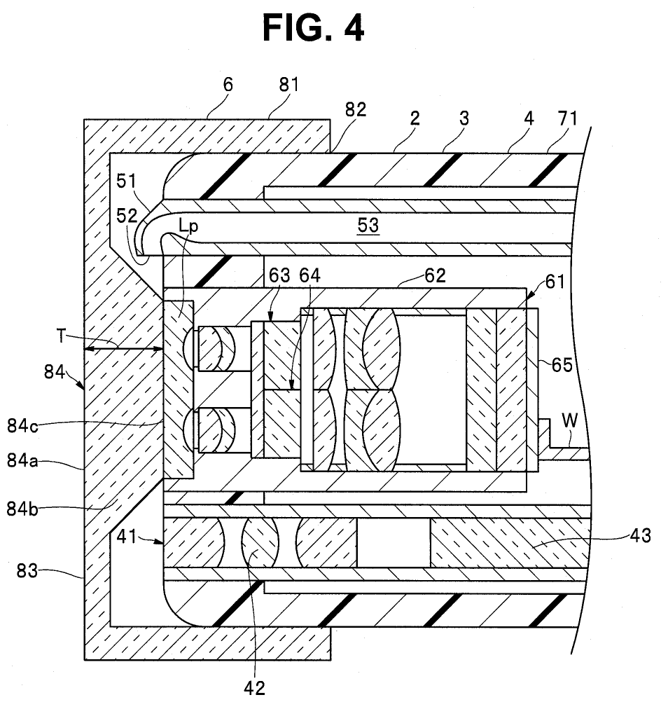

FIG. 4 is a schematic cross-sectional view showing an example of a state where the distal end hood 6 is attached to the distal end portion 4.

When the distal end portion 4 is inserted through the insertion port 82, the optical component Lp comes into contact with the emission surface 84c. When the distal end portion 4 is further pushed, the support member 83 is elastically deformed, and the light transmission body 84 is pushed in the distal end direction. As shown in FIG. 4, the distal end hood 6 is attached to the distal end portion 4 in a state where the emission surface 84c and the optical component Lp are in close contact with each other. The emission surface 84c and the optical component Lp are arranged to be parallel to each other.

The treatment instrument insertion hole 85 is arranged to face the treatment instrument insertion opening 21. The fluid feeding hole 86 is arranged to face the fluid feeding opening 31.

The nozzle 51 is arranged in the proximal end direction compared with the support member 83, and jets a fluid into a gap between the support member 83 and the distal end portion 4. The jetted fluid removes dirt on the distal end surface of the distal end portion 4 and on the proximal end surface of the support member 83, and is discharged to the outside from the treatment instrument insertion hole 85 and the fluid feeding hole 86.

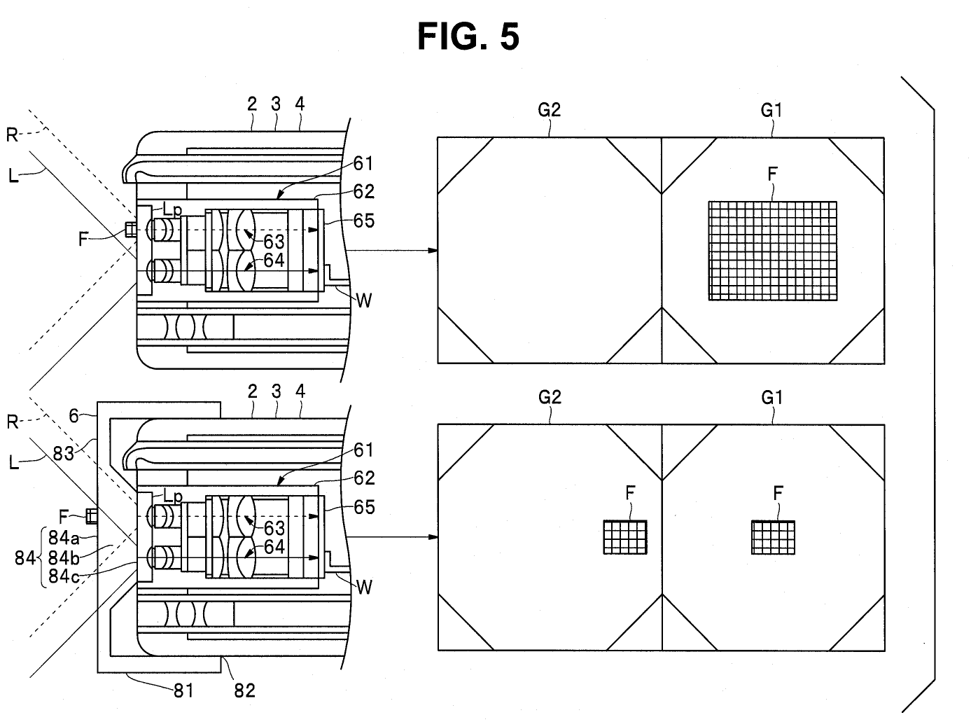

FIG. 5 is an explanatory diagram illustrating an example of the right-eye optical image G1 and the left-eye optical image G2.

When the illumination unit 41 irradiates the subject with the illumination light, the illumination light passes through the distal end hood 6 and illuminates the subject. The return light from the subject is incident on the incident surface 84a, and is emitted from the emission surface 84c to the optical component Lp. The right-eye optical image G1 obtained from a right-eye visual field R by the right-eye optical system 63 and the left-eye optical image G2 obtained from a left-eye visual field L by the left-eye optical system 64 are projected onto the image pickup device 65. The image pickup device 65 converts the right-eye optical image G1 and the left-eye optical image G2 into an image pickup signal, and outputs the image pickup signal to the video processor 12. The video processor 12 generates a stereoscopic image, and outputs the stereoscopic image to the monitor 14.

As shown in an upper part of FIG. 5, when a foreign matter F adheres to, for example, the distal end surface of the right-eye optical system 63 in a state where the distal end hood 6 is not attached, despite the foreign matter F is not reflected on the left-eye optical image G2, the foreign matter F is largely reflected on the right-eye optical image G1, and thus the stereoscopic image gives an unpleasant feeling to the user.

As shown in a lower part of FIG. 5, when the distal end hood 6 is attached, even if the foreign matter F adheres to the right-eye optical system 63 in the distal end direction, the foreign matter F is displayed small in both of the right-eye optical image G1 and the left-eye optical image G2 as examples, and the stereoscopic image becomes more natural.

The light transmission body 84 is more easily and reliably arranged in properly parallel with the optical component Lp by being brought into close contact with the optical component Lp. Due to the parallel arrangement with the optical component Lp, the light transmission body 84 prevents different distortions between the right-eye optical image G1 and the left-eye optical image G2.

According to the embodiment, the distal end hood 6 can be more easily and reliably attached to the endoscope 2 in the correct position to prevent the distortion given to the right-eye optical image G1 and the left-eye optical image G2 and to prevent the user's unpleasant feeling.

(Modification 1)

In the embodiment, the frame 81 is attached to the periphery of the outer tube 71 without a gap, but may be attached with a gap. In the embodiment, the support member 83 and the light transmission body 84 are made of resin, but may be made of glass.

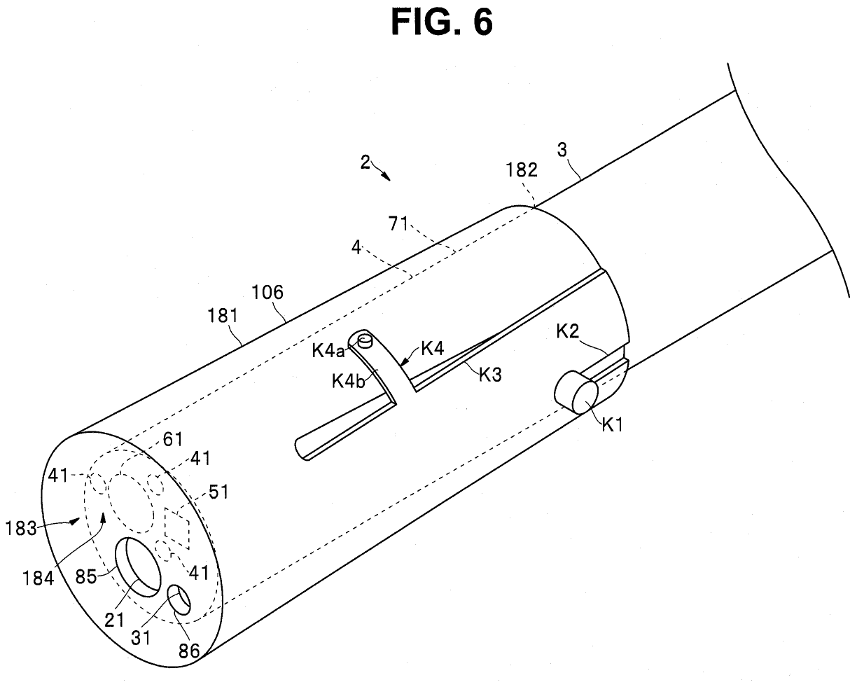

FIG. 6 is a perspective view showing an example of a distal end portion 4 and a distal end hood 106 of an endoscope 2 in an endoscope system 1 according to Modification 1 of the embodiment. In the present modification, the same components as the components of the embodiment and other modifications will not be described.

The endoscope 2 includes a positioning protrusion K1 on an outer peripheral surface of the distal end portion 4.

The distal end hood 106 includes a frame 181, an insertion port 182, a support member 183, a light transmission body 184, a positioning protrusion K1, a positioning groove K2, a slit K3, and a fixing portion K4.

The frame 181 is made of, for example, resin. The frame 181 has a tubular shape, and has an outer diameter larger by a predetermined amount than an outer diameter of the outer peripheral surface of the distal end portion 4 to form a gap with the outer peripheral surface of the distal end portion 4.

The insertion port 182 is provided at a proximal end of the distal end hood 106.

The support member 183 extends inward in the radial direction from an inner peripheral side of the frame 181.

The light transmission body 184 extends to the inside of the support member 183.

The support member 183 and the light transmission body 184 configure a filter.

The positioning protrusion K1 is provided to protrude from the outer peripheral surface of the distal end portion 4.

The positioning groove K2 is provided to be recessed in the distal end direction from the insertion port 182 of the distal end hood 106, and guides the positioning protrusion K1. A length and a position in a circumferential direction of the positioning groove K2 are adjusted in advance such that the distal end hood 106 is positioned at the distal end portion 4.

In other words, the positioning protrusion K1 and the positioning groove K2 configure a positioning portion. The positioning portion is provided on at least one of the distal end portion 4 and the distal end hood 106, and performs positioning of the insertion portion 3 and the distal end hood 106 such that the light transmission body 184 comes into parallel contact with the distal end surface of the optical component Lp.

The slit K3 is provided to be recessed in the distal end direction from the insertion port 182.

The fixing portion K4 is configured by, for example, a fixing protrusion K4a and a belt K4b arranged in the circumferential direction to intersect the slit K3 and locking the fixing protrusion K4a. The fixing portion K4 fixes an angle between the frame 181 and the light transmission body 184 when the distal end hood 106 is positioned and the light transmission body 184 comes into parallel contact with the distal end surface of the optical component Lp.

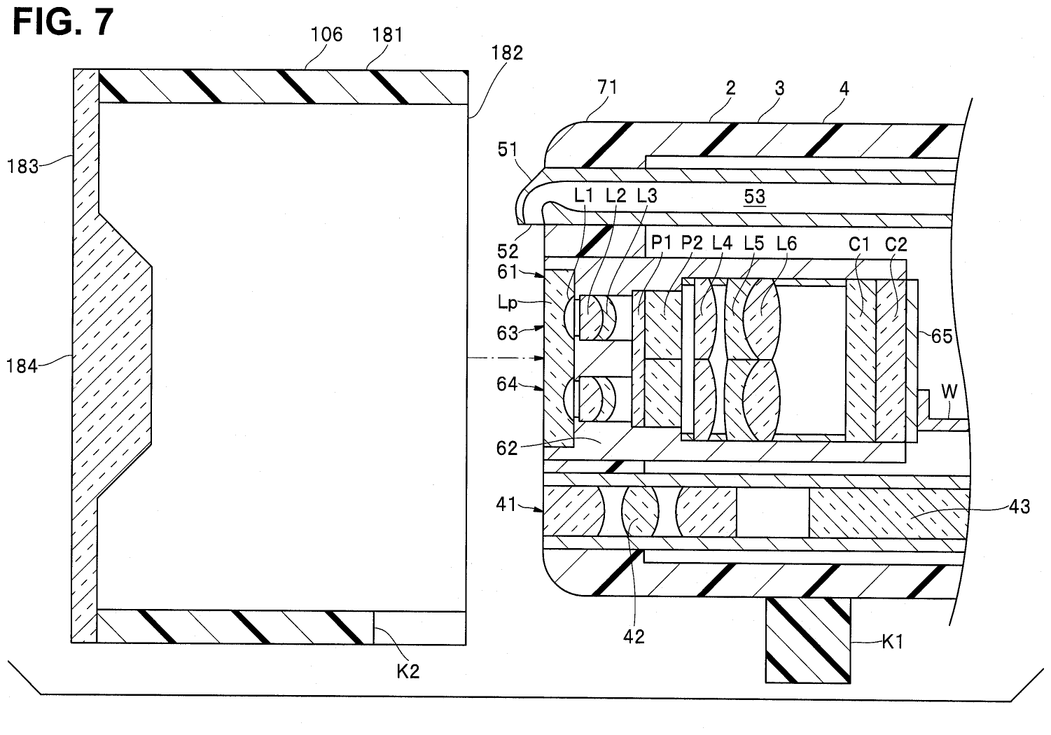

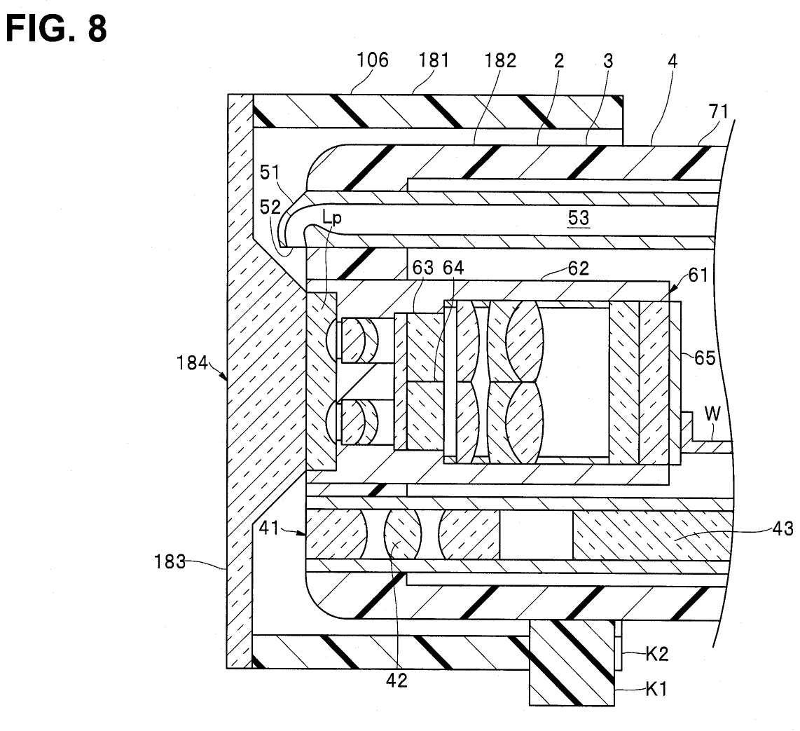

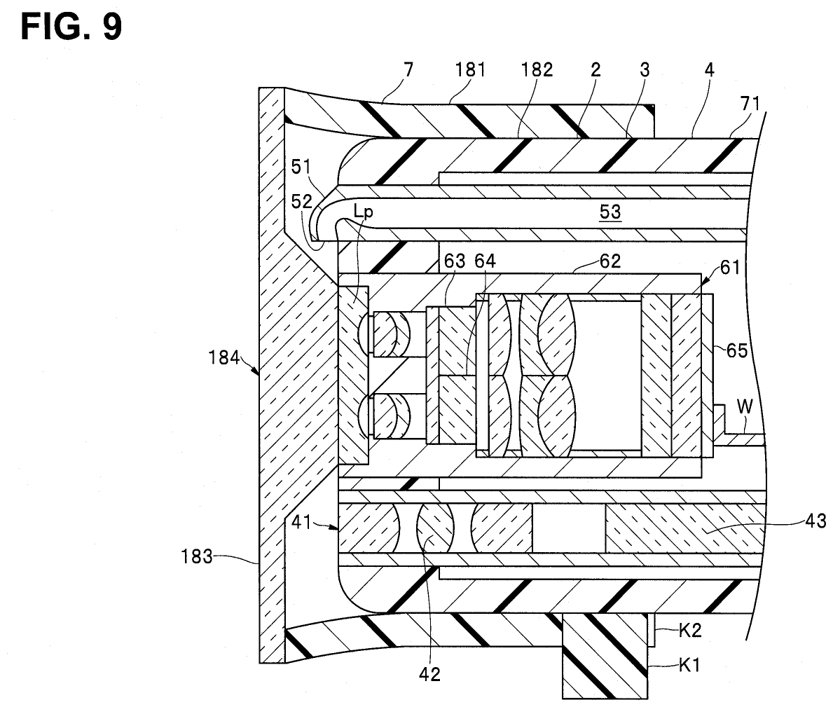

FIGS. 7, 8, and 9 are schematic cross-section views showing examples of the distal end portion 4 and the distal end hood 106 of the endoscope 2 in the endoscope system 1. FIG. 7 shows a state before the distal end hood 106 is attached, FIG. 8 shows a state where the distal end hood 106 is being attached, and FIG. 9 shows a state where the distal end hood 106 is attached.

As shown in FIG. 7, the distal end portion 4 is inserted from the insertion port 182 such that the positioning protrusion K1 is guided to the positioning groove K2.

As shown in FIG. 8, the distal end portion 4 is guided to the positioning groove K2, and is inserted until the emission surface 84c and the optical component Lp are in close contact with each other. When the emission surface 84c and the optical component Lp are in close contact with each other, the optical component Lp and the emission surface 84c become in parallel with each other.

As shown in FIG. 9, when the frame 181 narrows along the outer peripheral surface of the distal end portion 4 and the fixing protrusion K4a is locked by the belt K4b, the distal end hood 106 is fixed to the endoscope 2 (see FIG. 6).

Thus, the distal end hood 106 can more easily perform the positioning with the endoscope 2.

(Modification 2)

In the embodiment and Modification 1, the fluid delivered from the nozzle 51 is discharged from the treatment instrument insertion hole 85 and the fluid feeding hole 86, but may be drained from a drain port N.

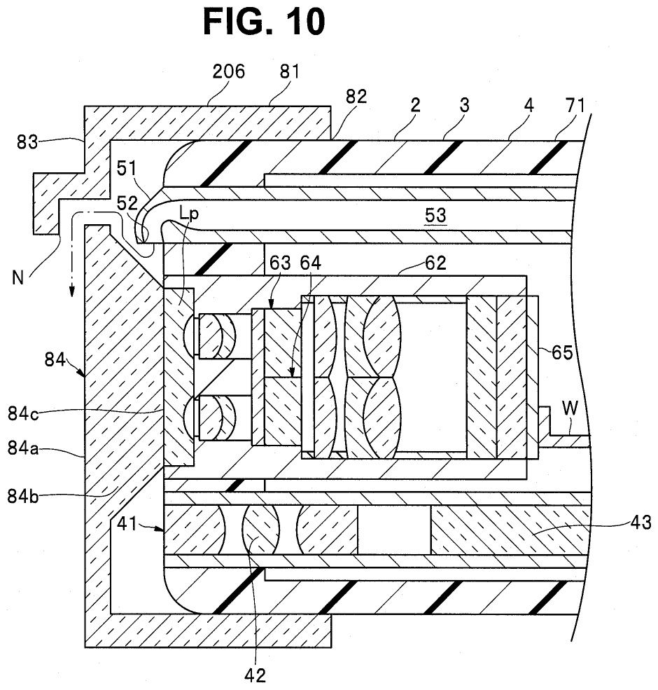

FIG. 10 is a schematic cross-sectional view showing an example of a distal end portion 4, a distal end hood 206, and a drain port N of an endoscope 2 in an endoscope system 1 according to Modification 2 of the embodiment of the present invention. In the present modification, the same components as the components of the embodiment and other modifications will not be described.

The distal end hood 206 includes the drain port N.

The drain port N is arranged to penetrate between the distal end surface and the proximal end surface of the support member 83, and the drain port N is arranged to face the incident surface 84a.

When a fluid is delivered from the nozzle opening 52, the fluid flows between the support member 83 and the distal end portion 4, and is discharged from the drain port N toward the incident surface 84a.

Thus, the incident surface 84a in the distal end hood 206 is cleaned by the fluid delivered from the nozzle opening 52.

(Modification 3)

In the embodiment and Modifications 1 and 2, the fluid delivered from the nozzle 51 flows between the distal end hood 6, 106, or 206 and the distal end portion 4, but may be guided to flow to the drain port N.

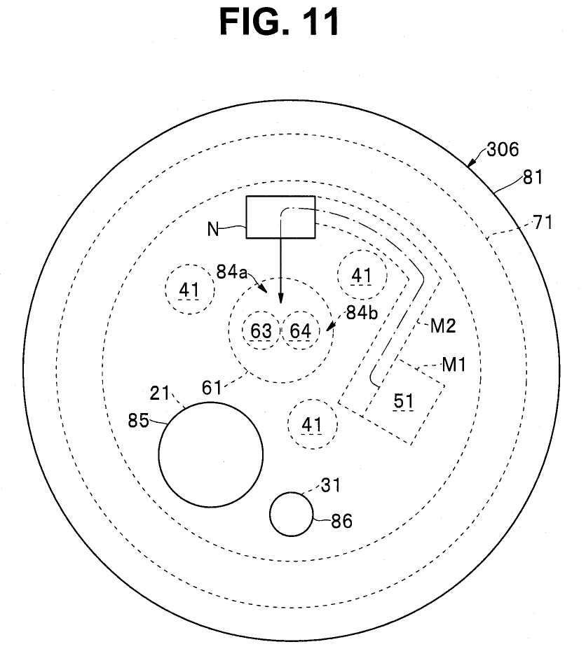

FIG. 11 is a front view showing an example of a distal end portion 4 and a distal end hood 306 of an endoscope 2 in an endoscope system 1 according to Modification 3 of the embodiment of the present invention.

In the present modification, the same components as the components of the embodiment and other modifications will not be described.

The distal end hood 306 includes a guide flow path M2. The support member 83 includes, on the proximal end side surface, a nozzle housing chamber M1 that houses the nozzle 51 and a guide flow path M2 that connects the nozzle housing chamber M1 and the drain port N.

When the distal end hood 306 is attached to the distal end portion 4, the nozzle housing chamber M1 and the guide flow path M2 can guide the fluid jetted from the nozzle 51 to the drain port N.

When the fluid is delivered from the nozzle opening 52 into the nozzle housing chamber M1, the fluid flows through the guide flow path M2 and is discharged from the drain port N toward the incident surface 84a.

In other words, the filter includes the guide flow path M2 through which the fluid jetted from the nozzle 51 is guided to a region behind the main body 84b of the light transmission body 84.

Thus, the distal end hood 306 can guide the fluid, which is delivered from the nozzle opening 52, to the drain port N.

In the embodiment and the modifications, the light transmission body 84 or 184 is in close contact with the optical component Lp, but the present invention is not limited thereto. For example, a transparent fluid such as a gel may be filled between the light transmission body 84 or 184 and the distal end surface of the optical component Lp, or an adhesive may be provided or a spacer may be arranged between the light transmission body 84 or 184 and the optical component Lp.

When the emission surface 84c and the optical component Lp are provided to be substantially and perfectly parallel to each other, a very small gap may be provided between the emission surface 84c and the distal end surface of the optical component Lp.

The support member 83 is configured such that at least the light transmission body 84 transmits light to the right-eye optical system 63 and the left-eye optical system 64 via the optical component Lp. Unless an image of the support member 83 is formed in the right-eye optical system 63 and the left-eye optical system 64, the support member 83 does not necessarily transmit light.

In the embodiment and modifications, the support member 83 or 183 is continuously provided on the distal end side of the frame 81 or 181, but a tubular member may be continuously provided from the distal end side of the frame 81 or 181 in the distal end direction such that the subject can be pressed.

The present invention is not limited to the embodiment described above, and various modifications and changes can be made without departing from the gist of the present invention.

What is claimed is:

1. An endoscope system comprising:
an endoscope; and
a distal end hood,
the endoscope including
an insertion portion configured to be inserted into a subject in a longitudinal direction,
a first optical system provided at a distal end portion of the insertion portion and configured to observe the subject,
a second optical system provided at the distal end portion to be adjacent to the first optical system and configured to observe the subject with parallax with respect to the first optical system,
an optical component provided on a distal end side of the first optical system and the second optical system and having light transmission properties, and
a protruding member that further protrudes in a distal end direction from a distal end surface of the distal end portion than the optical component,
the distal end hood including
a frame attached to a periphery of the distal end portion, and
a filter provided on an inner peripheral side of the frame, the filter including: a light transmission body provided so as to face a distal end surface of the optical component and having light transmission properties; and a support member provided at a non-contact position with the protruding member to support the light transmission body.

2. The endoscope system according to claim 1, wherein
the light transmission body has a shape that is in close contact with an entirety of the distal end surface of the optical component.

3. The endoscope system according to claim 2, wherein
the support member adjusts an angle between the frame and the light transmission body such that the light transmission body comes into parallel contact with the distal end surface of the optical component.

4. The endoscope system according to claim 3, further comprising
a fixing portion,
wherein the frame forms a gap with an outer peripheral surface of the distal end portion, and
the fixing portion fixes the angle between the frame and the light transmission body when the light transmission body comes into parallel contact with the distal end surface of the optical component.

5. The endoscope system according to claim 3, further comprising:
a positioning portion provided on at least one of the distal end portion and the distal end hood to perform positioning between the insertion portion and the distal end hood such that the light transmission body comes into parallel contact with the distal end surface of the optical component.

6. The endoscope system according to claim 3, wherein
the support member has elasticity to allow the positioning between the insertion portion and the distal end hood to be performed in a state where the light transmission body is in parallel contact with the distal end surface of the optical component.

7. The endoscope system according to claim 1, wherein
the protruding member includes a nozzle configured to jet a fluid onto the distal end surface of the distal end portion, and
the support member is provided at a non-contact position with a distal end of the nozzle when the light transmission body is attached to the distal end surface of the optical component.

8. The endoscope system according to claim 7, wherein
the filter includes a guide flow path that guides the fluid jetted from the nozzle to a region behind the light transmission body.

9. The endoscope system according to claim 1, wherein
a transparent fluid is filled between the light transmission body and the distal end surface of the optical component.

10. The endoscope system according to claim 1, wherein
the frame is externally fitted on an outer peripheral surface of the distal end portion.

11. A distal end hood comprising:
a frame attached to a periphery of a distal end portion of an endoscope; and
a filter provided on an inner peripheral side of the frame, the filter including: a light transmission body having light transmission properties and provided so as to face a distal end surface of an optical component of the endoscope provided on a distal end side of a first optical system and a second optical system having parallax with each other; and a support member provided at a non-contact position with a protruding member of the endoscope to support the light transmission body, the protruding member further protruding in a distal end direction from the distal end portion than the optical component.
